(19) United States
(12) Patent Application Publication
(10) Pub. No.: US 2017/0240968 A1
(43) Pub. Date: Aug. 24, 2017

(54) ALLELIC POLYMORPHISMS ASSOCIATED WITH REDUCED RISK FOR ALZHEIMER'S DISEASE

(71) Applicant: Brigham Young University, Provo, UT (US)

(72) Inventor: John S.K. KAUWE, Springville, UT (US)

(21) Appl. No.: 15/503,605

(22) PCT Filed: Aug. 12, 2015
(86) PCT No.: PCT/US2015/044864

§ 371 Date: Feb. 13, 2017

Related U.S. Application Data
(60) Provisional application No. 62/037,040, filed on Aug. 13, 2014.

(51) Int. Cl. C12Q 1/68; G01N 33/50

(52) U.S. Cl. CPC C12Q 1/6883; G01N 33/5023; C12Q 2600/156; C12Q 2600/106

(57) ABSTRACT

The present invention provides methods for determining an individual's risk of developing Alzheimer's disease. The methods include collecting a biological sample from an individual; genotyping a nucleic acid in the biological sample for a genetic polymorphism in a RAB10 gene or a SAR1A gene or both the RAB10 gene and the SAR1A gene, and determining from the genotyping a decreased risk of developing Alzheimer's disease when the genetic polymorphism in the RAB10 gene or the SAR1A gene or both the RAB10 gene and the SAR1A gene is present. The methods may also include determining the presence of SNP rs 142787485 which comprises an adenine (A) to guanine (G) change in the 3′ untranslated region of the RAB 10 gene, and/or determining the presence of SNP rs7653 which comprises a cytosine (C) to thymine (T) change in the 3′ untranslated region of the SAR1A gene.

### RELATED APPLICATIONS

This application claims the benefit of U.S. Provisional Application No. 62/037,040, filed Aug. 13, 2014, which is incorporated by reference herein in its entirety.

### FEDERALLY SPONSORED RESEARCH OR DEVELOPMENT

This invention was made with support under Grant No. R01-AG042611 from the National Institute of Health. The government has certain rights in this invention.

### SEQUENCE LISTING

The instant application contains a Sequence Listing which has been submitted electronically in ASCII format and is hereby incorporated by reference in its entirety. The ASCII copy, created on Aug. 11, 2015, is named 14706-60 sequencelisting_ST25.txt and is approximately 1 KB in size.

### BACKGROUND OF THE INVENTION

1. Field of the Invention

The present invention generally relates to a method for determining the risk of developing Alzheimer's disease. Further encompassed are methods for screening drugs for treatment of Alzheimer's disease and methods of treatment.

2. Description of the Related Art

Alzheimer's disease (AD) is the most common neurodegenerative disorder and affects nearly 6 million people in the United States alone. Alzheimer's disease is emotionally and financially devastating to those affected and their caretakers. The number of affected individuals is predicted to double in the next decade and there are currently no effective preventions or cures. There is an urgent need to develop the knowledge necessary to cure this terrible disease.

Recent progress is encouraging; international collaboration and skilled application of state of the art technology has led to the identification of several common alleles associated with Alzheimer's disease. Recent work using whole genome and whole exome datasets has identified rare variants with large protective and risk effects on Alzheimer's disease in amyloid precursor protein (APP), Apolipoprotein E (APOE), Phospholipase D3 (PLD3) and Triggering Receptor Expressed on Myeloid cells 2 (TREM2). The APOE gene on chromosome 19ql3 has been identified as a strong risk factor associated with Alzheimer's disease. For example, ApoE-ε4 is the high risk variant of the APOE gene, the gene most associated with increased risk for late-onset Alzheimer's disease. ApoE-ε4 refers to the presence of both SNPs rs7412 (C;C) and rs429358 (C;C).

Comparing with these rare, functional risk variants, characterization of biological mechanisms by which common variants modulate risk for the Alzheimer's disease has proven to be extremely difficult. Common protective variants may act by very different mechanisms than risk variants and are likely to represent much more tractable drug targets. Accordingly, a need exists for new methods of determining individuals' risk of developing Alzheimer's disease by identifying common variants with large protective effects on Alzheimer's disease. In addition, there is an ongoing need for methods of identifying individuals with decreased risk of developing Alzheimer's disease, which are especially useful for screening potential therapeutic drugs for the disease.

### SUMMARY OF THE INVENTION

The invention is based, in part, upon the discovery of two single nucleotide polymorphisms (SNPs) located in RAB10 and SAR1A genes that are significantly associated with reduced risk for Alzheimer's disease. Two single variants at two polymorphic sites, rs142787485 and rs7653, have been found to be associated with a reduced risk of developing AD as determined by statistical analysis, by virtue of linkage analysis, and/or by the virtue of the fact that they differ in frequency between case and control groups in association analysis.

Accordingly, in one aspect of the present invention, a method for determining an individual's risk of developing Alzheimer's disease is provided. The method includes collecting a biological sample from an individual; genotyping a nucleic acid in the biological sample for a genetic polymorphism in a RAB10 gene or a SAR1A gene or both the RAB10 gene and the SAR1A gene; and determining from the genotyping a decreased risk of developing Alzheimer's disease when the genetic polymorphism in the RAB10 gene or the SAR1A gene or both the RAB10 gene and the SAR1A gene is present. In some aspects, the RAB10 genetic polymorphism is a variation in the 3′ untranslated region (rs142787485). In some aspects, the RAB10 genetic polymorphism is an adenine to guanine change in rs142787485 (SEQ ID NO: 1). In some aspects, the SAR1A genetic polymorphism is a variation in the 3′ untranslated region (rs7653). In some aspects, the SAR1A genetic polymorphism is a cytosine to thymine change in rs7653 (SEQ ID NO: 2)

In some aspects, the individual's risk of developing Alzheimer's disease determined by methods provided by this invention can also be used to select a subject population for a clinical trial. In some aspects, the presence of the genetic polymorphism in the RAB10 gene or the SAR1A gene or both the RAB10 gene and the SAR1A gene indicates that the individual should be excluded from the clinical trial. In some aspects, individuals with the genetic polymorphism (rs142787485, SEQ ID NO: 1) in the RAB10 gene or the genetic polymorphism (rs7653, SEQ ID NO: 2) in the SAR1A gene or both is/are absent are selected for a therapeutic treatment for Alzheimer's disease.

In some aspects, the therapeutic treatment may comprise modulating expression or activity of RAB10, SAR1A or both RAB10 and SAR1A. In some aspects, the therapeutic treatment for Alzheimer's disease comprises administering an RNAi, an antisense oligonucleotide or antibody therapeutic treatment to modulate the expression of RAB10. In some aspects, the therapeutic treatment for Alzheimer's disease comprises administering an RNAi, an antisense oligonucleotide or antibody therapeutic treatment to modulate the expression of SAR1A.

In another aspect, a method of screening for biologically active agents that modulate the expression of RAB10 is provided. The method includes combining a candidate agent with a cell comprising a nucleotide sequence which does not include an allelic variant of the RAB10 gene comprising an adenine to guanine change in rs142787485 (SEQ ID NO: 1); and determining the effect of the agent upon the expression and/or activity of RAB10 relative to a control agent.

In another aspect, a method of screening for biologically active agents that modulate the expression of SAR1A is provided. The method includes combining a candidate agent with a cell comprising a nucleotide sequence which does not include an allelic variant of the SAR1A gene comprising a cytosine to thymine change in rs7653 (SEQ ID NO: 2); and determining the effect of the agent upon the expression and/or activity of SAR1A relative to a control agent.

In another aspect, a method for treating or preventing Alzheimer's disease is provided. The method comprises introducing a polymorphism comprising rs142787485 (SEQ ID NO: 1) into the RAB10 gene or introducing a polymorphism comprising rs7653 (SEQ ID NO: 2) into the SAR1A gene to a subject having Alzheimer's disease.

In yet another aspect, a kit for use in determining an individual's risk of developing Alzheimer's disease in a subject is provided. The kit includes a) a nucleic acid reagent for detecting an Alzheimer's disease-associated genetic polymorphism in a gene selected from a RAB10 gene and a SAR1A gene; and b) instructions for detecting the genetic polymorphism. In some aspects, the nucleic acid reagent in the kit may include a) a specific probe that detects a variation in the 3′ untranslated region (rs142787485) of RAB10 or a variation in the 3′ untranslated region (rs7653) of SAR1A; or b) an allele-specific primer or primer pair that provides for detection of a variation in the 3′ untranslated region (rs142787485) of RAB10 or a variation in the 3′ untranslated region (rs7653) of SAR1A.

Advantages of the present disclosure will become more apparent to those skilled in the art from the following description of embodiments that have been shown and described by way of illustration. The invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1A shows that multipoint linkage results from human Chromosome 2 in the first pedigree with statistical excess of AD deaths, showing a peak LOD score of 2.21 located between rs4341893 and rs2252032 on Chromosome 2 and includes 14898 markers and 101 genes.

FIG. 1B shows that multipoint linkage results from human Chromosome 10 in the second pedigree with statistical excess of AD deaths, showing a peak LOD score of 2.10 detected in two adjacent regions on Chromosome 10, which includes 10686 variants in 138 genes. These peaks are located between rs10823229 and rs7900882 and between rs7918631 and rs3740382.

FIG. 2 shows an immunoblot of RAB10 expression.

FIG. 3A shows ELISA data measuring Aβ42 (left), Aβ40 (middle) and Aβ42/Aβ40 ratio (right) panels in cells with RAB10 shRNA. Experiments and their respective controls (GFP for overexpression and Scrambled for shRNA) are depicted on Aβ42 (left), Aβ40 (middle) and Aβ42/Aβ40 ratio (right) panels. Each bar represents the results from six replicates.

FIG. 3B shows ELISA data measuring Aβ42/Aβ40 ratios in RAB10 expressing cells and cells expressing RAB10 shRNA. FIG. 3B shows the effects of Rab10 for Aβ42/Aβ40 ratio only. Each bar represents the results from 10 replicates.

FIG. 4 shows the results of association analysis presented in Table 1.

FIG. 5 shows the results of filtering of whole genome sequence data in linkage regions presented in Table 2.

### DETAILED DESCRIPTION OF THE PREFERRED EMBODIMENTS

As discussed previously, the invention is based, in part, upon the discovery of two single nucleotide polymorphisms (SNPs) located in RAB10 and SAR1A genes that are significantly associated with reduced risk for Alzheimer's disease. Two protective variants, A>G (rs142787485) in the RAB10 gene on human chromosome 2 and C>T (rs7653) in the SAR1A gene on human chromosome 10, have been found to be associated with a reduced risk of developing AD as determined by statistical analysis, linkage analysis, and/or by the virtue of the fact that they cluster with variants at polymorphic sites identified by association analysis. Individuals with one or more protective alleles have lower risk of developing AD when compared to those with the respective common alleles.

### Definitions

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms.

The term “single polynucleotide polymorphism (SNP)” refers to a DNA sequence variation that involves a change in a single nucleotide. They present in humans with a frequency of about once in every 1000 bases and contribute to differences among individuals. The majority of SNPs have no effect. However, some affect the risk for certain diseases.

The term “genotype” refers to a description of the alleles of a gene or genes contained in an individual or a sample. Diploid individuals have a genotype that comprises two different sequences (heterozygous) or one sequence (homozygous) at a polymorphic site. As used herein, no distinction is made between the genotype of an individual and the genotype of a sample originating from the individual. Although typically a genotype is determined from samples of diploid cells, a genotype can be determined from a sample of haploid cells, such as a sperm cell.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “genetic marker” as used herein refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two alleles.

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs.

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes.

The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides, conservatively modified variants thereof, complementary sequences, and degenerate codon substitutions that have similar binding properties as the reference nucleic acid and are metabolized in a manner similar to naturally occurring nucleotides.

The terms “diagnose” and “diagnosis” refer to the ability to determine or identify whether an individual has a particular disorder (e.g., a condition, illness, disorder or disease), for example, the Alzheimer's disease. The term “prognose” or “prognosis” refers to the ability to predict the course of the disease (including to predict the risk of developing the disease) and/or to predict the likely outcome of a particular therapeutic or prophylactic strategy.

The term “screen” or “screening” as used herein has a broad meaning. It includes processes intended for diagnosing or for determining the susceptibility, propensity, risk, or risk assessment of an asymptomatic subject for developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various polymorphic variants disclosed herein (and typically other polymorphic variants and genetic or behavioral characteristics) so as to determine where the individual lies on the spectrum of disease risk-neutrality-protection. A sample such as a blood sample may be taken from the individual for purposes of conducting the genetic testing using methods known in the art or yet to be developed. Alternatively, if a health provider has access to a pre-produced data set recording all or part of the individual's genome (e.g. a listing of polymorphic variants in the individual's genome), screening may be done simply by inspection of the database, optimally by computerized inspection. Screening may further comprise the step of producing a report identifying the individual and the identity of alleles at the site of at least one or more of the rs142787485 and rs7653 SNPs.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers may be present within the same gene or gene cluster.

The terms “risk,” “protective,” and “neutral” are used to characterize the phenotypic impact of allelic variations, e.g., SNPs, haplotypes, and diplotypes. A “risk allele”, “risk SNP, or “risk haplotype” is an allelic form of a genetic locus that includes at least one variant polymorphism, or two or more variant alleles, associated with an increased risk for developing AD. The term “variant” refers to a nucleotide sequence in which the sequence differs from the sequence most prevalent in a defined population. A variant polymorphism can be in the coding or non-coding portions of the gene. A “protective allele, “protective SNP”, or “protective haplotype” is an allelic form of a genetic locus that includes at least one variant polymorphism, and preferably a set of variant polymorphisms, associated with decreased risk of developing AD and thus can “protect” the subject by reducing its propensity for the disease.

The terms “patient,” “subject” and “individual” are used interchangeably herein, and mean a mammalian (e.g., human) subject to be treated and/or to obtain a biological sample from.

The term “LOD score”, or logarithm of odds score, is a statistical test used in genetic linkage analysis. The LOD score compares the probability of obtaining the test data if the two loci are linked to the probability of obtaining the test data if the two loci are not linked.

The present invention discloses the associations of polymorphic sites rs142787485 in the RAB10 gene and rs7653 in the SAR1A gene with the risk of developing AD, which were not known. The SNPs are by nature comprised in an isolated polynucleotide or nucleic acid molecule, corresponding to SEQ ID NO: 1-2.

RAB10, a member of the RAS oncogene family, includes 12 exons and spans just under 104 kilobases on chromosome 2p23.3. RAB10 is located in the plasma membrane and plays a critical role in the secretory pathway (Mitra et al., 2011). Specifically, RAB10 is involved in regulation of membrane trafficking and movement of proteins from the golgi apparatus to the membrane (Hutagalung et al., 2011; Bao et al., 1998). RAB10 also has a role in neurotransmitter release, phagosome maturation and Glut4 translocation (Mitra et al., 2011). RAB10 is expressed in the brain. In neurons, RAB10 is involved in axonogenesis through regulation of vesicular membrane trafficking toward the axonal plasma membrane (English et al., 2013). RAB10 is known to bind APP and RNAi silencing of RAB10 is known to decrease Aβ levels without affecting sAPPBeta levels, possibly by altering gamma secretase cleavage or changing secretion/degradation of AB (Udayar et al., 2013; Olah et al., 2011).

SEQ ID NO: 1 indicates the position of polymorphism rs142787485 in its surrounding sequence. Rs142787485 is an A to G change that occurs in the 3′ untranslated region of the RAB10 gene. CTCAGCTCAACTGCATTTCAGTTGTGTTATAGTCCAGTTCTTATCAACA TT (SEQ ID NO: 1) (Forward Strand). There are no previous reports of association with phenotypes in the NHGRI GWAS database (Welter et al., 2014). The SNP results in a gain of miRNA regulation via the miRNAs MIR374A and MIR374B. While MIR 374A is not highly expressed in brain, MIR374B shows substantial expression in multiple brain regions (Hawrylycz et al., 2012). The rs142787485 variant results in a change in miRNA regulation and a likely change in expression. This change in expression may result in decreased abeta, a known mechanism for reducing AD risk.

SAR1A is a member of the small GTPase superfamily and includes 25 exons and spans just over 23 kilobases on chromosome 10q22.1. SAR1A is located in the endoplasmic reticulum and is involved in transport from the endoplasmic reticulum to the Golgi apparatus (Watson et al., 2006). SAR1A is known to bind APP and is expressed in the brain (Hawrylycz et al., 2012; Olah et al., 2011).

SEQ ID NO: 2 indicates the position of polymorphism rs7653 in its surrounding sequence. GTGTTAGTTTTCTTAATTTTTTTTTTCCCCTATTTGTCCCTTGTCACTTTG (SEQ ID NO: 2) (Reverse Strand) Rs7653 is located in the 3′ untranslated region of the SAR1A gene and is a change from C to T in the reverse strand. There is no known or predicted functional consequence of this variant and no previous reports of association with phenotypes in the NHGRI GWAS database (Welter et al., 2014).

Aspects of the present invention encompass an approach including careful selection of unique phenotypes and pedigrees followed by linkage analysis. Other aspects of the invention include whole-genome sequencing to interrogate the linkage region and identify candidate SNP variants. Additionally, aspects of the present invention may include a large-scale association study in an independent series of unrelated individuals to validate the association of each candidate variant identified with reduced risk for Alzheimer's disease.

In one embodiment, the method of determining an individual's risk of developing Alzheimer's disease includes genotyping a nucleic acid in the biological sample of an individual for a genetic polymorphism in a RAB10 gene or a SAR1A gene or both the RAB10 gene and the SAR1A gene; and determining from the genotyping a decreased risk of developing Alzheimer's disease when the genetic polymorphism in the RAB10 gene or the SAR1A gene or both the RAB10 gene and the SAR1A gene is present. In some embodiment, the alleles of the two SNPs may be inferred by genetic testing of other markers or by measuring the levels of activity or concentration of the proteins or corresponding RNA expression levels of RAB10 and/or SAR1A. In some embodiments, the method of determining an individual's risk of developing Alzheimer's disease may also be used to aid in the diagnosis or prognosis of AD in an individual.

In some embodiments, the SNP genotyping assays can be performed with TaqMan® 5′-nuclease assay or custom Custom TaqMan® SNP Genotyping Assays. Custom allele-specific TaqMan® MGB probes containing a PCR primer pair may be used to detect specific SNP targets (rs142787485 and rs7653 SNPs).

A method for screening for a therapeutic agent effective to modulate the expression of RAB10 is also provided. The method includes combining a candidate agent with a cell comprising a nucleotide sequence which does not include an allelic variant of the RAB10 gene comprising an adenine to guanine change in rs142787485 (SEQ ID NO: 1); and determining the effect of the agent upon the expression and/or activity of RAB10 relative to a control agent. Suitable therapeutic agents identified in the screen can inhibit cell deterioration associated with AD. These methods also includes incubating neuronal cells in vitro with an agent to be tested, measuring RAB10 activity directly or indirectly, comparing the activity to RAB10 activity in the absence of the agent to be tested, and identifying the therapeutic agent by detection of modulation of RAB10 activity in the presence of the agent.

Examples of measuring RAB10 activity include measuring mRNA level, mRNA transcription rate, protein level, assaying the activity of the RAB10 protein, and measuring activity of a reporter gene operably linked to a RAB10 regulatory element. Suitable therapeutic agents will modulate the expression of RAB10 relative to a control agent.

A method for screening for a therapeutic agent effective to modulate the expression of SAR1A is also provided. The method includes combining a candidate agent with a cell comprising a nucleotide sequence which does not include an allelic variant of the SAR1A gene comprising a cytosine to thymine change in rs7653 (SEQ ID NO: 2); and determining the effect of the agent upon the expression and/or activity of SAR1A relative to a control agent. Suitable therapeutic agents identified in the screen can inhibit the cell deterioration associated with AD. The methods also includes incubating neuronal cells in vitro with an agent to be tested, measuring SAR1A activity directly or indirectly, comparing the activity to SAR1A activity in the absence of the agent to be tested, and identifying the therapeutic agent by detection of modulation of SAR1A activity in the presence of the agent. Examples of measuring SAR1A activity include measuring mRNA level, mRNA transcription rate, protein level, assaying the activity of the SAR1A protein, and measuring activity of a reporter gene operably linked to a SAR1A regulatory element. Suitable therapeutic agents will modulate the level of SAR1A relative to a control agent.

In some embodiments, a non-human transgenic animal or mammalian cell based assay (preferably a human cell based assay) with a genome including the human RAB10 gene that does not include the haplotype identified by the SNP SEQ ID NO: 1 may be used in a screening assay. In some embodiments a non-human transgenic animal or mammalian cell based assay (preferably a human cell based assay) with a genome including the human SAR1A gene that does not include the haplotype identified by the SNP SEQ ID NO: 2 may be used in a screening assay. The non-human animal or mammalian cell based assay can be used as a disease model for screening therapeutic compounds for treatment of AD. Thus, the disclosure provides a method for screening for a therapeutic agent effective to inhibit development of cell deterioration associated with dementia. The method may also include administering to the transgenic animal an agent to be tested for its effectiveness in treating or preventing the cell deterioration associated with dementia, and assessing the effectiveness of the agent.

In some embodiments, genome editing techniques may be used to provide a model for screening therapeutic compounds or for methods of treatment. Non-limiting examples of genome editing techniques include targeted cleavage events, for example, to induce targeted mutagenesis, induce targeted deletions of cellular DNA sequences, and facilitate targeted recombination at a predetermined chromosomal locus. In some embodiments, specific nucleases such as engineered zinc finger nucleases (ZFN), transcription-activator like effector nucleases (TALENs), or using the CRISPR/Cas system with an engineered crRNA/tracr RNA (‘single guide RNA’) may be used to guide specific cleavage. In yet other embodiments, viral or other recombinant vectors may be used.

In some embodiments, the recombinant vectors incorporating DNA sequences for human RAB10 gene that does not include the haplotype identified by the SNP SEQ ID NO: 1 and/or the human SAR1A gene that does not include the haplotype identified by the SNP SEQ ID NO: 2. The nucleic acid segments used in the present invention, regardless of the length of the coding sequence itself, can be combined with other DNA or RNA sequences, such as promoters, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. A “heterologous” sequence refers to a sequence that is foreign or exogenous to the remaining sequence. A heterologous gene refers to a gene that is not found in nature adjacent to the sequences with which it is now placed. A method for treating or preventing Alzheimer's disease is also provided. In some embodiments, the method includes introducing an isolated nucleotide sequence encoding a RAB10 gene polymorphism comprising rs142787485 (SEQ ID NO: 1) or an isolated nucleotide sequence encoding a SAR1A gene polymorphism comprising rs7653 (SEQ ID NO: 2) or both to a subject having Alzheimer's disease. In certain embodiments, the method may include introducing the nucleotide sequence as a vector and expressing an isolated nucleotide sequence encoding a RAB10 gene polymorphism comprising rs142787485 (SEQ ID NO: 1) or an isolated nucleotide sequence encoding a SAR1A gene polymorphism comprising rs7653 (SEQ ID NO: 2) or both in a subject having Alzheimer's disease.

In certain embodiments, one or more oligonucleotides of the invention are provided in a kit or on device (e.g., an array) useful for detecting the presence of a predisposing or a protective polymorphism in a nucleic acid sample of an individual whose risk for AD is being assessed. A useful kit can contain a nucleic acid reagent specific for detecting an Alzheimer's disease-associated genetic polymorphism in a gene selected from a RAB10 gene and a SAR1A gene as well as instructions for detecting the genetic polymorphism for their use to determine risk for AD.

In some embodiments, the nucleic acid reagent may be oligonucleotides in a form suitable for use as a specific probe that detects a variation in the 3′ untranslated region (rs142787485) of RAB10 or a variation in the 3′ untranslated region (rs7653) of SAR1A, for example, fixed to an appropriate support membrane. In other embodiments, the nucleic acid reagent can be an allele-specific primer or primer pair intended for use as amplification primers for providing for detection of a variation in the 3′ untranslated region (rs142787485) of RAB10 or a variation in the 3′ untranslated region (rs7653) of SAR1A.

As yet another alternative, a useful kit can contain antibodies to a protein that is altered in expression levels, structure and/or sequence when the SNP rs142787485 and/or rs7653 are present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

In one embodiment, a kit or device for diagnosing susceptibility to Alzheimer's disease in a subject comprising a nucleic acid reagent for detecting an Alzheimer's disease-associated genetic polymorphism in a gene selected from a RAB10 gene and a SAR1A gene. The nucleic acid reagent may distinguish alleles at least one polymorphic site selected from the group consisting rs142787485 and rs7653. In an exemplary embodiment, the nucleic acid reagent includes primers for amplification of a region spanning a polymorphic site rs142787485 or rs7653.

As will be proved further below in the methods and results section, the SNPs or the corresponding isolated polynucleotides can then be applied to a new model or method for determining the genetic risk that an individual may present for AD, and a kit for implementing the method. The following examples serve to illustrate the invention without limiting the invention in its scope.

### EXAMPLES

### Example 1

### Methods

### Linkage Samples

The control population for linkage analysis consists of APOE ε4 carriers over age 75 who remain non-demented after clinical assessment was identified from the Cache County Study. The Cache County Study was initiated in 1994 to investigate the association of APOE genotype and environmental exposures on cognitive function and dementia. This cohort of 5,092 Cache County, Utah, residents (90% of those aged 65 or older), has been followed continually for over 15 years, with four triennial waves of data collection and additional clinical assessments for those at high-risk for dementia. DNA samples were obtained from 97.6% of participants. The Cache County population is exceptionally long-lived and ranked number one in life expectancy among all counties in the 1990 U.S. Census (Murray et al., 1998). All members of the Cache County Study have been linked to the Utah Population Database (UPDB) and their extended genealogies are known. This population was the source of most of the Centre d′Etude du Polymorphisme Humain (CEPH) families that have been used as representative of Caucasians in countless genetic studies worldwide, including the HapMap project. Each control has been sequenced with a mean of 40× coverage across the entire genome. In addition, each sample was genotyped using the Illumina 2.5M SNP Array.

The Alzheimer's disease case population consists of a subset of Cache County Study participants that have been carefully ascertained as part of the Cache County Dementia Progression Study. Since 2002, persons identified with incident dementia from the Cache County Study have been followed prospectively in this progression study. This study examines factors that affect the rate of dementia progression in cognitive, functional, and neuropsychiatric symptom trajectories. Together with the Cache County Study, this project has collected data on 581 incident Alzheimer's disease cases; the majority of these have been followed to their deaths (492 have a “complete” course). An expert panel of neurologists, neuropsychologists, geropsychiatrists, and a cognitive neuroscientist assigned final diagnoses of dementia following standard research protocol, e.g. NINCDS-ADRDA criteria for Alzheimer's disease (Breitner et al., 1999) or NINCDS-AIREN criteria for vascular dementia (Roman et al., 1993). Each case was genotyped using the Illumina 2.5M SNP Array.

### Pedigree Selection

The UPDB is a population-based resource linking the computerized genealogy of the Utah pioneers and their descendants to various electronic health data repositories for the state, including Utah death certificates (Skolnick et al., 1980). The UPDB includes over 6 million individuals, 2.4 million of which have at least 3 generations of genealogy data and who are part of the original Utah pioneer data; over 1 million of these individuals have at least 12 of their 14 immediate ancestors in the genealogy. Using this database, large pedigrees were identified that include at least 4 samples from our control population and 4 samples from our case population.

### Pedigree Analysis

Utah death certificates from 1904 to the present have been coded and record-linked to individuals in the UPDB allowing consideration of all relationships among individuals who have a cause-of-death that includes Alzheimer's disease. Alzheimer's disease, as a coded cause-of-death, first appeared in the International Classification of Disease (ICD) Revision 9, and is also present in revision 10. Only individuals to have died from Alzheimer's disease if their death certificate ICD codes included Alzheimer's disease as a primary or contributing cause-of-death were considered. Pedigrees with a statistically significant excess of deaths from Alzheimer's disease were identified using the UPDB. All descendants of a set of UPDB founders, who have a death certificate, are considered a pedigree. The expected number of Alzheimer's disease deaths among the deceased descendants is calculated by applying the cohort-specific Alzheimer's disease death rates to the numbers of descendants in each cohort. The ratio of observed to expected Alzheimer's disease deaths among descendants was used to test the hypothesis of a significant excess of deaths within each pedigree.

### SNP Genotyping/Linkage Analysis

SNPs from the OmniExpress chip, reduced to a set of high heterozygosity markers with low/no pair-wise linkage disequilibrium, were used to conduct linkage analyses to identify shared chromosomal segments among our controls. With the use of MCLINK (Thomas et al., 2000), different modes of inheritance were considered and corrected for multiple tests (Camp, 2001). Monte Carlo Markov Chain methodology using blocked Gibbs sampling to provide haplotype reconstructions to extract inheritance information in pedigrees was used (Thomas et al., 2000; Camp et al., 2001; Abkevich et al., 2001). For parametric analyses, the program calculates robust multi-point LOD scores (TLODs). Our linkage analyses utilize a set of parametric and non-parametric statistical methods to evaluate evidence for linkage to particular genomic regions. Our experience with prior successful linkage studies has confirmed the robustness of these models for TLOD analyses. Only LOD scores >1.86 (corresponding to a false-positive rate of 1 per genome) were considered as suggestive evidence for linkage, and scores >3.30 as significant, as defined by Lander and Kruglyak (1995) (Lander et al., 1995), whether considering evidence for one pedigree or for multiple pedigrees. An aggregated pedigree disequilibrium test (PDT) that can incorporate large complex pedigrees and allow aggregation of rare variants over an arbitrary genomic window was used (Martin et al., 2000). Once linkage evidence is established via these methods, all SNP markers in the region to provide fine mapping localization evidence will be used. Linkage evidence from each pedigree is considered independently.

### Whole Genome Sequence Variant Filtering

Variants were analyzed using the Ingenuity Variant Analysis and Tute Genomics analysis software. For the Ingenuity Variant Analysis we used version 3.0.20140422 with content versions as follows: Ingenuity Knowledge Base (Arrakis 140408.002), COSMIC (v68), dbSNP (Build 138 (Aug. 9, 2013)), 1000 Genome Frequency (v3), TargetScan (v6.2), EVS (ESP6500 0.0.21), JASPAR (Oct. 12, 2009), PhyloP hg18 (11/2009), PhyloP hg19 (01/2009), Vista Enhancer hg18 (Oct. 27, 2007), Vista Enhancer hg19 (Dec. 26, 2010), CGI Genomes (11/2011), SIFT (01/2013), BSIFT (01/2013), TCGA (Sep. 5, 2013), PolyPhen-2 (HumVar Training set 2011_12), Clinvar (Feb. 11, 2014).

Variants in the suggestive linkage regions from each pedigree were filtered as follows. Variants with the following characteristics were kept:

1) Call quality at least 20.0 in cases or at least 20.0 in controls and outside top 0.2% most exonically variable 100 base windows in healthy public genomes (1000 genomes) and outside top 1.0% most exonically variable genes in healthy public genomes (1000 genomes);

2) Associated with gain of function or heterozygous or hemizygous or haploinsufficient or compound heterozygous and occur in at least 1 of the control samples at the gene level in the control samples;

3) Experimentally observed to be associated with a phenotype: Pathogenic, Possibly Pathogenic or established gain of function in the literature or inferred activating mutations by Ingenuity or predicted gain of function by BSIFT or in a microRNA binding site or Frameshift, in-frame indel, or stop codon change or Missense and not predicted to be innocuous by SIFT or disrupt splice site up to 2.0 bases into intron or deleterious to a microRNA or structural variant or in promoter binding site or in enhancer or in evolutionary-conserved region with a phyloP p-value of less than or equal to 0.01 or in untranslated region;

4) Are within 2 hops upstream and that are known or predicted to affect: susceptibility to late-onset Alzheimer disease, sporadic Alzheimer disease (non-familial Alzheimer's disease), Alzheimer disease-5 (late-onset familial Alzheimer's disease), Alzheimer disease-2 (Alzheimer disease type 2), Alzheimer's disease type 4, late-onset Alzheimer disease (late-onset Alzheimer's disease) or genes within 1 hop downstream of them.

Variants were excluded that are observed with an allele frequency greater than or equal to 3.0% of the genomes in the 1000 genomes project or greater than or equal to 3.0% of the public Complete Genomics genomes or greater than or equal to 3.0% of the NHLBI ESP exomes (European American).

### Association Analyses

Association Samples and Genotyping: the cohort for association analysis consists of 435 AD cases and 801 elderly controls, neuropathologically and clinically confirmed, originating from the UK and North America. The mean age at disease onset was 67 years (range 36-94 years) for cases and the mean age of ascertainment was 79 years (range 56-103 years) for controls (shown in Table 1).

### Exome Sequencing

Whole exome sequencing was performed on a discovery cohort of 590 controls and 435 AD cases. DNA was extracted from blood and brain for cases and brain only for controls, using standard protocols. Library preparation for next generation sequencing used DNA (between 1 μg and 3 μg) fragmented in a Covaris E210 (Covaris Inc.). Following fragmentation, DNA was end-repaired by 5′phosphorylation, using the Klenow polymerase. A poly-adenine tail was added to the 3′end of the phosphorylated fragment and ligated to Illumina adapters. After purification using an AMPure DNA Purification kit (Beckman Coulter, Inc), adapter-ligated products were amplified. The DNA library was then hybridized to an exome capture library (NimbleGen SeqCap EZ Exome v2.0, Roche Nimblegen Inc. or TruSeq, Illumina Inc.) and precipitated using streptavidin-coated magnetic beads (Dynal Magnetic Beads, Invitrogen). Exome-enriched libraries were PCR-amplified, and then DNA hybridized to paired-end flow cells using a cBot (Illumina, Inc.) cluster generation system. Samples were sequenced on the Illumina HiSeg™ 2000 using 2×100 paired end reads cycles.

### Whole Genome Sequencing

Genome sequencing was performed in 211 elderly, clinically healthy controls, from the Cache County Study on Memory in Aging. All samples were sequenced with the use of Illumina HiSeq technology. Alignment was performed with the use of CASAVA software and variant calling was performed with the use of SAMtools21 and GATK (McKenna et al., 2010; Li et al., 2009; Hosseini et al., 2010.)

### Sequence Alignment and Variant Calling

Each of the samples in our dataset consisted of paired-end 100 base pair reads. The Burrows-Wheeler Aligner (BWA) (Li et al., 2009) was used to map the reads to build of the human genome (hg19/GRCh37). Following read mapping, we used SAMtools (Li et al., 2009), Picard (http://picard.sourceforge.net), and the Genome Analysis Toolkit (GATK) (McKenna et al., 2010; DePristo et al., 2011) to refine the resulting alignments by removing duplicates, performing realignment around InDels, and recalibrating base quality scores. The GATK's UnifiedGenotyper was then used to identify sequence variants, and subsequently filtered the variants and recalibrated variant quality scores (DePristo et al., 2011). The final dataset consisted of variant call format (VCF) files containing variants that passed all filters. Since the dataset consisted of a mix of exomes captured using different kits, and whole genome sequences, a highly conservative approach was employed to variant selection to increase our confidence that analyzed variants are true positives. The dataset of variants was limited to only those genomic regions we expected to have been sequenced in each of the exomes (based on capture probes used for exome library preparation) and whole genomes. Next, a list of all the variants present in at least a single sample was compiled. Each of the variants from the list of total variants in each sample was examined, whether or not the variant was called by the GATK, and reassigned the genotype for that variant according to the following criteria. (1) If the variant was called by the GATK and passed all filters, the GATK genotype was used. (2) If no variant was called at the genomic position in question, we returned to the raw VCF file and if there were reads containing the variant, but the variant was not called because of failing filters or because only a small number of reads contain the variant, we set the genotype to missing for the sample. (3) Finally, if all the reads at this position for the sample indicated reference alleles, we set the genotype to homozygous reference. Resulting sequence files were converted to Plink format (Purcell et al., 2007) using VCFTools (Danecek et al., 2011).

### Association Analyses

Association between rs14278748 (RAB10) and rs7653 (SAR1A) and AD status were tested using a linear regression after adjustment for age, gender, and PC's from stratification analysis using PLINK (Purcell et al., 2007). Given the linkage results, all tests were conducted under the specific assumption of a protective effect on AD status. For gene-based tests of association we used VAAST. We used the Variant Annotation Analysis and Search Tool (VAAST) to specifically test whether SAR1A and RAB10 are potential Alzheimer's disease genes. VAAST's primary purpose is to identify genes with damaging alleles using amino acid substitutions and allele frequency differences between a background population and the test population (Yandell et al., 2011); however, VAAST can also test for protective alleles in the same manner. We followed VAAST's best practices (available per request to the Yandell Lab) comparing 152 Alzheimer's disease cases from ADNI and 211 controls from the Cache County Study on Memory and Aging.

### Results

### Linkage Analysis

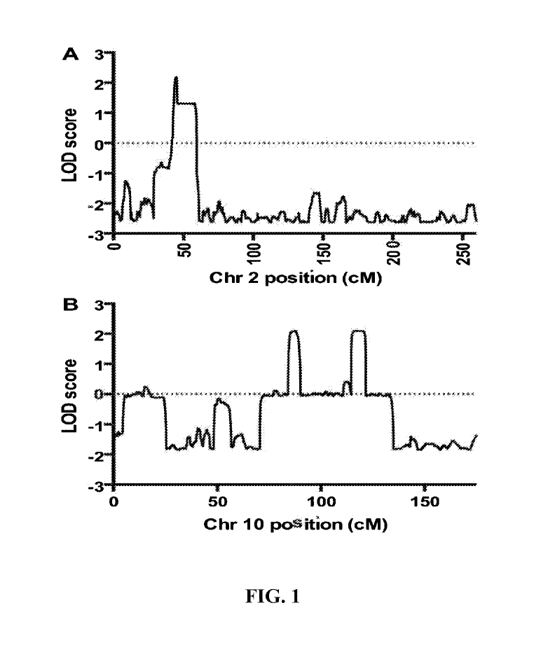

Five pedigrees that met our filtering criteria (statistical excess of AD deaths, inclusion of at least 4 samples each from our control and case populations) were identified. Linkage analysis yielded suggestive LOD scores for two of these pedigrees (FIG. 1A, 1B). In the first pedigree, a LOD score of 2.21 was detected on Chromosome 2. This peak is located between rs4341893 and rs2252032 on chromosome 2 and includes 14898 markers and 101 genes. In the second pedigree, a LOD score of 2.10 was detected in two adjacent regions on Chromosome 10, which includes 10686 variants in 138 genes. These peaks are located between rs10823229 and rs7900882 and between rs7918631 and rs3740382. We failed to detect suggestive or significant evidence of linkage in the three other pedigrees that were analyzed. Analysis of whole genome sequence for the likely sharers of these linked regions using the filtering described in the methods resulted in three markers for the chromosome 2 linkage region and 5 markers for the chromosome 10 linkage region (Table 2).

Due to limited power in our replication series we selected one marker from each region. Based on careful evaluation of the literature and functional annotations of each marker we selected rs142787485 (RAB10) from the chromosome 2 region and rs7653 (SAR1A) from the chromosome 10 region for follow-up in our replication series. To reduce multiple test burden, the other markers that survived filtering were not tested for association with AD status.

### Associations

Significant association with AD status for both rs142787485 and rs7653 were detected (Table 3). Gene based tests conducted using VAAST also resulted in significant associations for RAB10 (p=2.81E-05) and SAR1A (P=3.66E-06).

Table 3: significant association with AD status for both rs142787485 and rs7653

| | | | All Samples MAF |
|---|---|---|---|
| Gene | p-value | OR | Controls/Cases |
| RAB10 | 0.01836 | 0.5853 | 0.04135/0.02759 |
| SAR1A | 0.004947 | 0.3534 | 0.03/0.009195 |

### Example 2

Differential expression of RAB10 changes the Aβ42/Aβ40 ratio and influences Aβ42 production.

Methods

Plasmids

The plasmids used for this study were the following: pCMV6-Rab10 (Origene # RC201464) for the overexpression experiments and pGFP-V-RS-Rab10 shRNA (Origene #TG501823). Four shRNA versions per gene were tested to obtain greater knockdown levels.

Cell Culture

Mouse neuroblastoma cells (N2A/APP695) expressing human APP-695 isoform were kindly given by Celeste Karch, Ph.D. N2A/APP695 are a mouse neuroblastoma line that express human APP695 isoform and is commonly used in functional APP studies (Thinakaran, Teplow et al. 1996, Rajendran, Honsho et al. 2006, Wang et al. 2006). N2A/APP695 cells were plated and grown in Dulbecco's modified eagle medium (DMEM) and Opti-MEM (1:1) supplemented with 1% L-glutamine, 5% FBS and 1% anti-mycotic solution. Cells were grown between 80% to 90% confluence for posterior analyses. Upon confluency, cells were transiently transfected using Lipofectamine 2000 (Invitrogen). Culture media was changed 24 h after transfection. Following 48 h after transfection, cell media was collected and centrifuged for 10 minutes at 4° C. and protease inhibitor was added for peptide preservation. Cell pellets were collected, lysed and centrifuged with protease inhibitor to collect total protein. Protein concentration was measured using a BCA method in preparation for immunoblotting.

Transfection and Reporter Gene Assays

Functional assays of reporter gene constructs were performed by transient transfection of N2A/APP695 cells using Lipofectamine 2000 reagent (Life Technologies). Cells were allowed to grow to confluence between 85 to 90% and transfected with a pCDNA control vector to bring total DNA concentration 1.0 μg. Cells were growth for 48 h; following this time, media was collected for ELISA assays. Cells were washed and RNA was isolated from cells for RT-qPCR or lysed to assess protein concentration by Western blot analysis.

RNA Isolation and RT-PCR Analysis

In order to assess greatest knockdown, total RNA was isolated from N2A/APP695 cells after transfection with four plasmids containing specific shRNA for knockdown of RAB10 genes. RNA was extracted from cells 48 h following transfection using RNeasy (Qiagen) following manufacturer protocol. RNA was converted to cDNA using High-capacity cDNA reverse transcription (ABI). Following RT-PCR, Taqman real time PCR assays were used to observe the expression of RAB10 (Mm00489481_m1) from ABI technologies. Total gene expression was quantified in triplicates using an ABI-7900 Real-Time PCR system. A housekeeping gene GAPDH was used for normalizing expression values using the CT method.

Protein Analysis

N2A/APP695 cell lysates were used to assess RAB10 and APP695 protein expression by SDS-PAGE and Western blot analysis with primary mouse 9E10 or 6E10 polyclonal antibody and goat-anti mouse polyclonal as secondary antibody. Briefly, equivalent amounts of total protein were evaluated by SDS-PAGE, blocked with 5% nonfat milk, and exposed to the primary antibody diluted at 1:5000 at 4° C. overnight. Exposure to the secondary antibody was carried at 1:2000 for 2 h. Blots were exposed with enhanced chemiluminescence (Lumigen TMA-6).

Enzyme-Linked Immunosorbent Assay

The levels of Aβ40 and Aβ42 were measured from collected cell culture media by sandwich ELISA as described by the manufacturer (Invitrogen). ELISA values were obtained (pg/mL) and corrected for total intracellular protein (ug/mL) based on BCA measurements.

Statistical Analysis

Values are expressed as mean±SD obtained from at least three separate experiments in each group. Data were assessed by one-way analysis of variance (ANOVA). When ANOVA indicated significant differences, the Student's t-test was used with Bonferroni correction for multiple comparisons. Results presented are representative and those with P values <0.05 were considered significant.

Results

RAB10 Expression does not Affect Full-Length APP

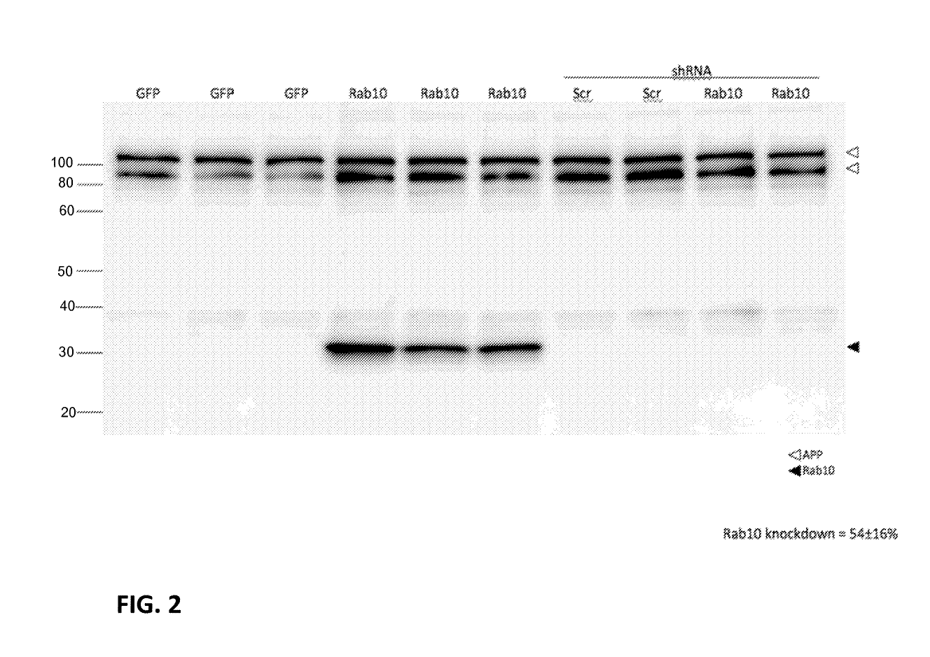

We sought to first test the effects of cellular RAB10 overexpression on full length APP. We transfected pCMV6-Rab10 and pCMV6-SAR1A overexpression plasmids in N2A/APP695 mouse cells. At 48 h after transfection, cells were harvested and protein expression was measured using 6E10 and 9E10 antibodies as described in methods section. FIG. 2 shows that full-length APP expression is the same in GFP control lanes with RAB10 transfected cells. We further tested whether this observation by immunoblotting with three different gradients of protein aliquots (25 ug/uL, 37.5 ug/uL and 50 ug/uL). The results were the same as before, indicating that APP and RAB10 exposure increase proportionally to the total protein immunoblotted (not shown). Similarly, we measured APP expression of transfected N2A/APP695 cells with shRNA plasmids that produced about a 54% knockdown of Rab10. FIG. 2 also shows no visible change in full length APP between the scrambled plasmid and the knockdown plasmids. Together, these results show that full length APP is not affected by overexpression of RAB10.

RAB10 expression alters Aβ levels.

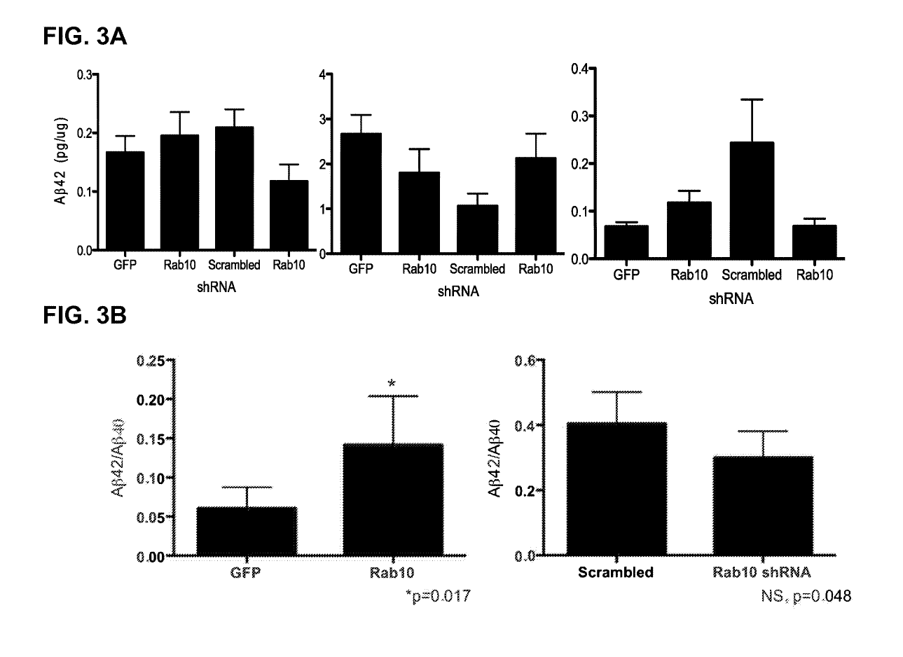

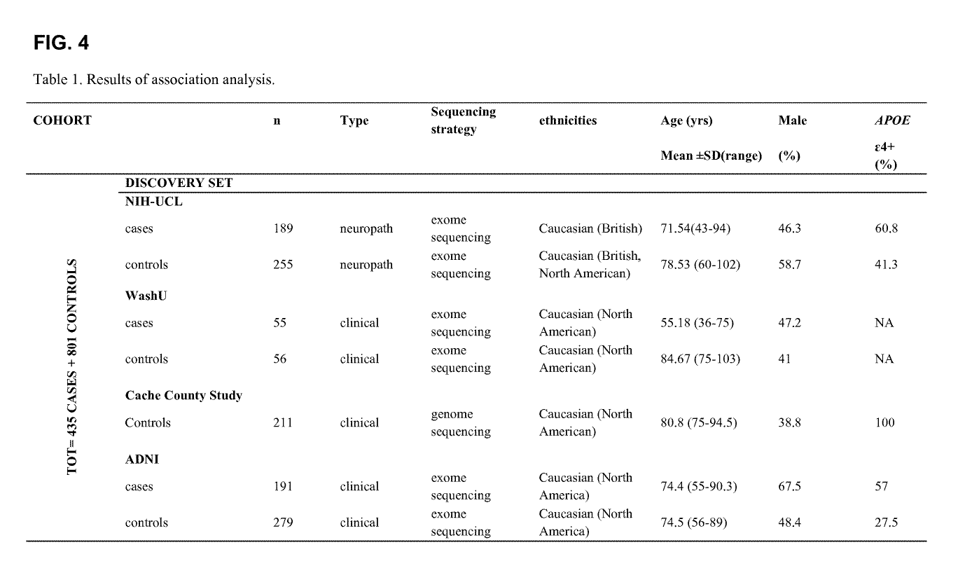

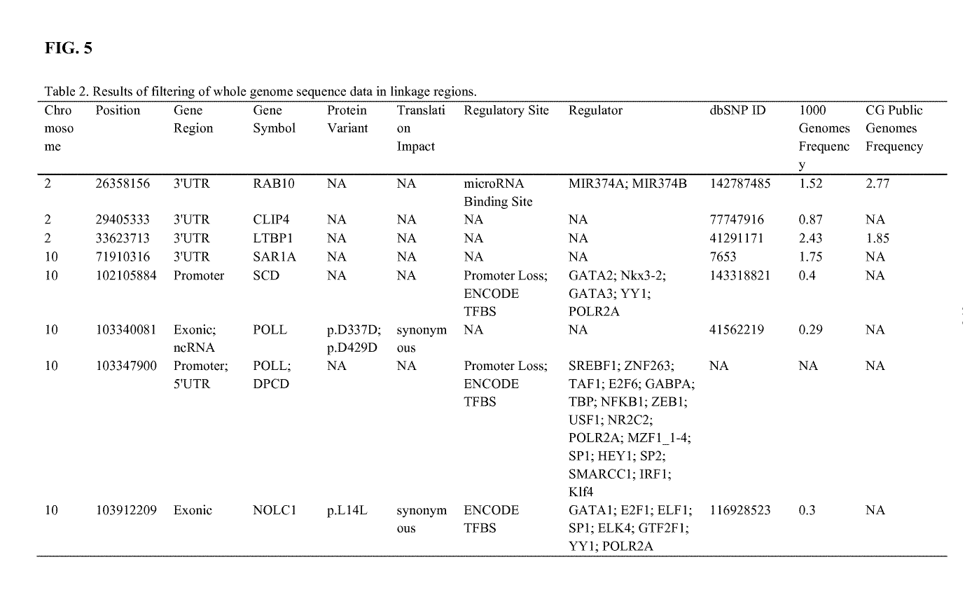

Next, we sought to quantify the effect of varying expression of RAB10 on Aβ levels. A sandwich ELISA measuring Aβ40 and Aβ42 levels was performed from cell media obtained from transient transfected cells with overexpression and shRNA plasmids for RAB10. We observed increased levels of Aβ42 levels and an increased Aβ40:42 ratio (FIG. 3A, p=0.0017) in cells overexpressing RAB10. Conversely, we observed the opposite effect in knockdown RAB10 cells, resulting in decreased Aβ42 levels and a decreased Aβ40:42 ratio (FIG. 3B), however, the p-value was verily significant p=0.048. Together, these results show that RAB10 plays a role in APP processing but not on full-length expression.

The practice of the present invention will employ, unless otherwise indicated, conventional techniques of molecular biology, immunology, microbiology, cell biology and recombinant DNA, which are within the skill of the art. See e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); the series METHODS IN ENZYMOLOGY (Academic Press, Inc.); PCR 2: A PRACTICAL APPROACH (Current Edition); ANTIBODIES, A LABORATORY MANUAL and ANIMAL CELL CULTURE (R. I. Freshney, ed. (1987)). DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Fundamental Virology, 2nd Edition, vol. I & II (B. N. Fields and D. M. Knipe, eds.)

Although the invention herein has been described in connection with a preferred embodiment thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

1. A method of detecting a genetic polymorphism, the method comprising:
collecting a biological sample from an individual;
genotyping a nucleic acid in the biological sample for a genetic polymorphism in a 3′ untranslated region of a RAB10 gene, the 3′ untranslated region of the RAB10 gene comprising SEQ ID NO: 1 or a 3′ untranslated region of a SAR1A gene, the 3′ untranslated region comprising SEQ ID NO: 2 or both of the nucleotide sequences of the 3′ untranslated region of the RAB10 gene and the SAR1A gene.

2. (canceled)

3. The method according to claim 1, wherein the RAB10 genetic polymorphism is an adenine to guanine change in SEQ ID NO: 1.

4. (canceled)

5. The method according to claim 1, wherein the SAR1A genetic polymorphism is a cytosine to thymine change in SEQ ID NO: 2.

6. The method according to claim 5, further comprising using the information to select a subject population for a clinical trial.

7. The method according to claim 6, wherein the presence of the genetic polymorphism in the RAB10 gene or the SAR1A gene or both the RAB10 gene and the SAR1A gene indicates that the individual should be excluded from the clinical trial.

8. The method according to claim 1, selecting the individual for a therapeutic treatment when the genetic polymorphism SEQ ID NO: 1 in the RAB10 gene or the genetic polymorphism SEQ ID NO: 2 in the SAR1A gene or both is or are absent.

9. The method according to claim 8, wherein the therapeutic treatment comprises modulating expression or activity of RAB10, SAR1A or both RAB10 and SAR1A.

10. The method according to claim 9, wherein the therapeutic treatment comprises administering an RNAi, an antisense oligonucleotide or antibody therapeutic treatment to modulate the expression of RAB10.

11. The method according to claim 9, wherein the therapeutic treatment comprises administering an RNAi, an antisense oligonucleotide or antibody therapeutic treatment to modulate the expression of SAR1A.

12. A method of screening for biologically active agents that modulate the expression of RAB10, the method comprising:
providing a cell identified as comprising a nucleotide sequence which does not include an allelic variant of the RAB10 gene comprising an adenine to guanine change in SEQ ID NO: 1;
combining a candidate agent with a cell; and
determining the effect of the agent upon the expression and/or activity of RAB10 relative to a control agent.

13. A method of screening for biologically active agents that modulate the expression of SAR1A, the method comprising:
providing a cell identified as comprising a nucleotide sequence which does not include an allelic variant of the SAR1A gene comprising a cytosine to thymine change in SEQ ID NO: 2;
combining a candidate agent with a cell; and
determining the effect of the agent upon the expression and/or activity of SAR1A relative to a control agent.

14. A method for treating or preventing Alzheimer's disease, comprising introducing a polymorphism comprising SEQ ID NO: 1 into the RAB10 gene or
introducing a polymorphism comprising SEQ ID NO: 2 into the SAR1A gene to a subject having Alzheimer's disease.

15. (canceled)

16. (canceled)

17. The method according to claim 13, further comprising using the information to select a subject population for a clinical trial.

18. The method according to claim 17, wherein the presence of the genetic polymorphism in the RAB10 gene or the SAR1A gene or both the RAB10 gene and the SAR1A gene indicates that the individual should be excluded from the clinical trial.


SEQUENCE LISTING (2 sequences)

SEQ ID NO: 1 - DNA, 51 bp, Artificial Sequence
  RAB10 rs142787485
ctcagctcaactgcatttcagttgtgttatagtccagttcttatcaacatt

SEQ ID NO: 2 - DNA, 51 bp, Artificial Sequence
  SAR1A rs7653
gtgttagttttcttaattttttttttcccctatttgtcccttgtcactttg